(19) United States
(12) Patent Application Publication
(10) Pub. No.: US 2023/0210691 A1
(43) Pub. Date: Jul. 6, 2023

(54) Contact Lens Tool

(71) Applicant: Spencer Obie, Houston, TX (US)

(72) Inventor: Spencer Obie, Houston, TX (US)

(21) Appl. No.: 18/146,892

(22) Filed: Dec. 27, 2022

Related U.S. Application Data
(63) Continuation-in-part of application No. 29/822,152, filed on Jan. 6, 2022.
(60) Provisional application No. 63/296,416, filed on Jan. 4, 2022.

(51) Int. Cl. A61F 9/00; B25J 1/04

(52) U.S. Cl. CPC A61F 9/0061; B25J 1/04

(57) ABSTRACT

The contact lens tool is a device intended to make it easier to insert and remove a contact lens from a person's eye. To accomplish this, the device includes a unique applicator head specifically designed to pick up and hold a contact lens. According to a preferred embodiment, the applicator head can be adjusted to various positions, depending on how the device is used. For inserting or removing a contact lens, the applicator head is most effective when partially open, forming a “Y” shape. This shape allows the applicator head to hold a wide range of contact lenses. Alternatively, the applicator head can be fully opened, forming a “T” shape. When not in use, the applicator head can be folded to a fully closed position, forming an “I” shape. This shape reduces space, allowing the device to be stored in a small compartment such as a wallet or purse.

### FIELD OF THE INVENTION

The present invention relates generally to a device capable of inserting and removing a contact lens from one's eye. More specifically, the present invention is a tool with a handle and an applicator head designed to make it easier for the user to insert and remove a contact lens from their eye.

### BACKGROUND OF THE INVENTION

There are many benefits associated with contact lens wear, including improved visual acuity for those with high prescriptions, an expanded visual field, improved contrast sensitivity, the ability to wear a variety of sunglasses for those with light-sensitivities, improved convenience, and improved perceived cosmesis. There are also several occupational and safety benefits that contact lens wear sometimes offers over spectacle lens wear. In addition, there are new medical technological innovations that use contact lenses as a platform to benefit an even larger number of people. However just as there are many benefits, there are also some risks to consider. Where glasses are low maintenance and can easily be put on and taken off, contact lenses require preparation and the proper technique to insert. Many people struggle considerably with handling contact lenses well enough to insert the contact lenses and may struggle equally or more so with removing the contact lenses. Proper mastery of the skill of contact lens insertion and removal can determine if the wearer will be able to continue to successfully wear contact lenses without complications. The complications of contact lens wear include eye infections, dry eye, and other eye irritations. Touching the eye is a major culprit for these complications, yet it is an essential part of the insertion and removal process. Furthermore, the majority of contact lens wearers do not wash their hands adequately before handling contact lenses, touching the eye, or touching the areas around the eye. This leads to the introduction of pathogens and other irritants to the eye, areas around the eye, and the contact lenses. Unfortunately, poor technique will likely lead to more touching causing the wearer to experience some of the other unpleasant contact lens complications and even avoid contact lenses altogether.

The two basic types of contact lenses can be categorized as hard contact lenses and soft contact lenses. The insertion and removal methods for each type of contact lens are different. One may typically use one's fingertips for the insertion and removal of both types, but hard contact lenses can be effectively removed with a plunger mechanism. The plunger mechanism is not effective for soft contact lenses, however. If used, the plunger mechanism could result in both the soft contact lens and the plunger mechanism becoming stuck to one's eye. A tweezer mechanism can potentially be used for the removal of soft contact lenses, although the tweezer mechanism is not always effective and can harm the corneal surface.

It is an objective of the present invention to provide a solution to the aforementioned problems. The present invention is a single instrument capable of inserting and removing a contact lens from one's eye. With regard to removal, the present invention can remove soft contact lenses in a manner, unlike the tweezer mechanism described above. Thus, the present invention eliminates the need for one to directly touch one's eyes during the insertion or removal of contact lenses, thereby preventing the difficulty and discomfort of said action, as well as reducing the risk of infection and other adverse events.

### SUMMARY

It is an aim of the present invention to make it easier to insert and remove a contact lens from one's eye. The present invention comprises a handle and an applicator head. In one embodiment, the entire device is made from a rigid, non-bendable material such as polycarbonate. In a preferred embodiment, the entire device is made from a soft and bendable material such as PVC. In the preferred embodiment, the applicator head can be adjusted to various positions, depending on how the device is used. For inserting or removing a contact lens, the applicator head is most effective when adjusted partially open in the shape of a “Y”. This allows the applicator head to hold a wide variety of contact lenses. If necessary, the applicator head can be adjusted fully open in the shape of a “T”. When not in use, the applicator head can be adjusted fully closed in the shape of an “I”. This shape reduces overall space, allowing the device to be stored in a wallet or purse. Due to the “I” shape's low profile, multiple devices can be stored together in a matchbox-like configuration allowing for increased portability. In addition, this matchbox configuration allows multiple devices to easily slip into various product packaging without altering their fundamental packaging.

### BRIEF DESCRIPTION OF THE DRAWINGS

FIG. 1 is a top-front perspective view of the present invention.

FIG. 2 is a diagram view of the present invention, showing a contact lens attached.

FIG. 3 is a front elevational view of the present invention.

FIG. 4 is a left-side elevational view of the present invention.

FIG. 5 is a top-front perspective view of the present invention in accordance with another embodiment.

FIG. 6 is a front elevational view of the present invention in accordance with another embodiment.

FIG. 7 is a front elevational view of the present invention in accordance with another embodiment.

FIG. 8 is a front elevational view of the present invention in accordance with another embodiment.

FIG. 9 is a top-front perspective view of the present invention, shown installed in a container.

### DETAIL DESCRIPTIONS OF THE INVENTION

All illustrations of the drawings are for the purpose of describing selected versions of the present invention and are not intended to limit the scope of the present invention.

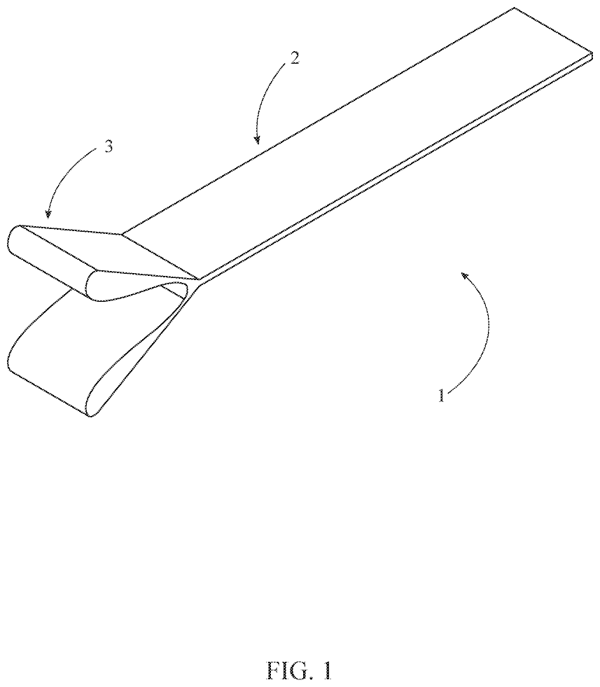

In reference to FIG. 1 through FIG. 9, the present invention is a contact lens tool 1. It is an aim of the present invention to allow the contact lens wearer (user) to insert and/or remove a contact lens 4 without the need to directly touch the anterior surface of the cornea 5. More specifically, the present invention is a contact lens tool 1 designed to hold the contact lens 4 in place as the user begins to insert or remove the contact lens 4 to or from the cornea 5. To hold the contact lens 4 in place, the contact lens tool 1 primarily relies on hydrostatic forces (e.g., surface tension). In a preferred embodiment, the contact lens tool 1 relies on hydrostatic forces, as well as a fluid phenomenon known as capillary action. Capillary action provides additional handling support. Capillary action is a dynamic mechanism that occurs when a liquid comes into contact with a porous material. In particular, the liquid naturally flows into the narrow spaces of the porous material without the assistance of, or even in opposition to, any external forces like gravity. The effect can be seen, for example, in the drawing up of liquids in a piece of paper or between the bristles of a paint brush. This effect occurs because of the intermolecular forces between the liquid and the surrounding solid surfaces within the porous material. More specifically, if the space within the porous material is sufficiently small, the liquid traverses into the small space, even upward against gravity, because the liquid is attracted to the solid surfaces surrounding the small space.

The following description is in reference to FIG. 1 through FIG. 9. As seen in FIG. 1, the contact lens tool 1 further comprises a handle 2 and an applicator head 3. The combination of the handle 2 and the applicator head 3 helps facilitate the insertion and removal of the contact lens 4.

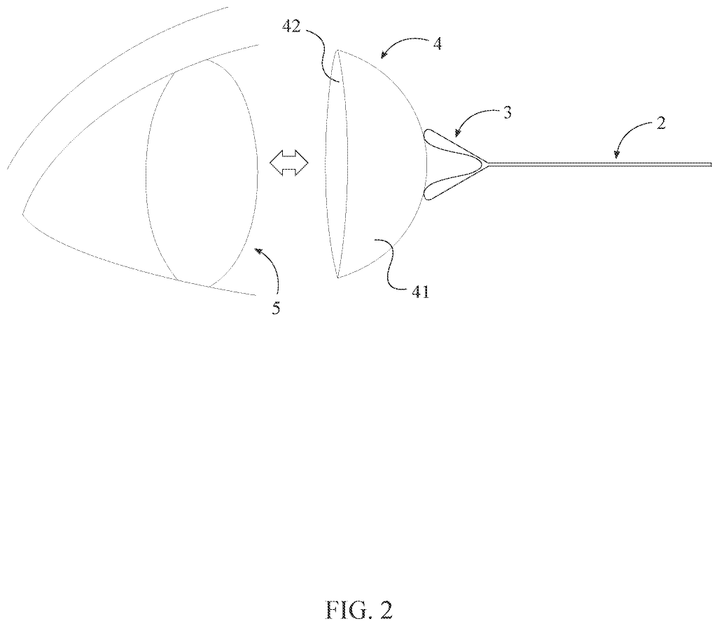

In the preferred embodiment, for insertion of the contact lens 4, the user begins by grasping the handle 2 between the user's pointer finger and thumb. As best seen in FIG. 2, the user then places the anterior surface 41 of the contact lens 4 onto an interior surface 33 of the applicator head 3. When wet, the capillary action described above allows the contact lens 4 to adhere to the applicator head 3. More specifically, the interior surface 33 of the applicator head 3 holds the anterior surface 41 of the contact lens 4 in place through the mild fluid interactions in the small spaces between the applicator head 3 and the contact lens 4. The mild fluid interactions of the liquid gently bind the contact lens 4 to the applicator head 3, even overpowering the effects of gravity. With the contact lens 4 in place, the user then lifts the contact lens tool 1 and inserts the contact lens 4 into the cornea 5. The subsequent fluid dynamic and force created between the posterior surface 42 of the contact lens 4 and the cornea 5 is greater than the adhesion force between the anterior surface 41 of the contact lens 4 and the interior surface 33 of the applicator head 3. Because of these weak fluid interactions, the contact lens tool 1 can be gently pulled away from the anterior surface 41 of the contact lens 4, as the contact lens 4 is now securely inserted into the user's cornea 5. The method described above can be repeated for the other eye as well.

For removal of the contact lens 4, the user begins by gently pressing the applicator head 3 against the contact lens 4 still resting on the cornea 5. The forces from the mild fluid interactions along with a gentle pressure applied downward cause the contact lens 4 to slide off the curvature of the cornea 5. As the contact lens 4 slides off the cornea 5 and sclera to reach the fornix, the suction of the contact lens 4 on the cornea 5 breaks, thereby dislodging the contact lens 4 from the user's eye and enabling the safe removal of the contact lens 4. As best seen in FIG. 2, the user can then pick up and remove the now loose contact lens 4 with the contact lens tool 1. Alternatively, the user can safely pluck the dislodged contact lens 4 using their fingers. Lastly, the user can apply contact lens solution to the contact lens 4 or perform any other necessary actions to properly maintain the contact lens 4. The method described above can be repeated for the other eye as well.

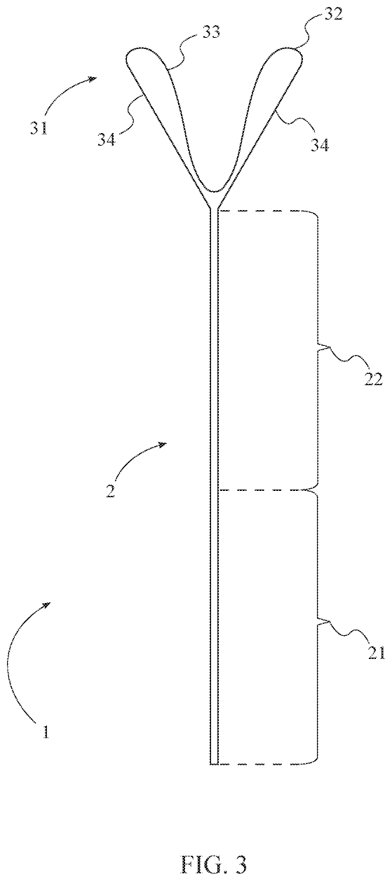

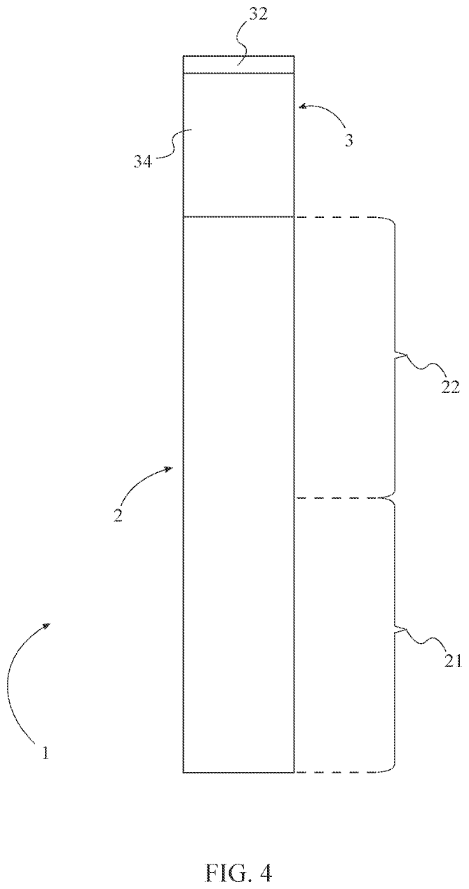

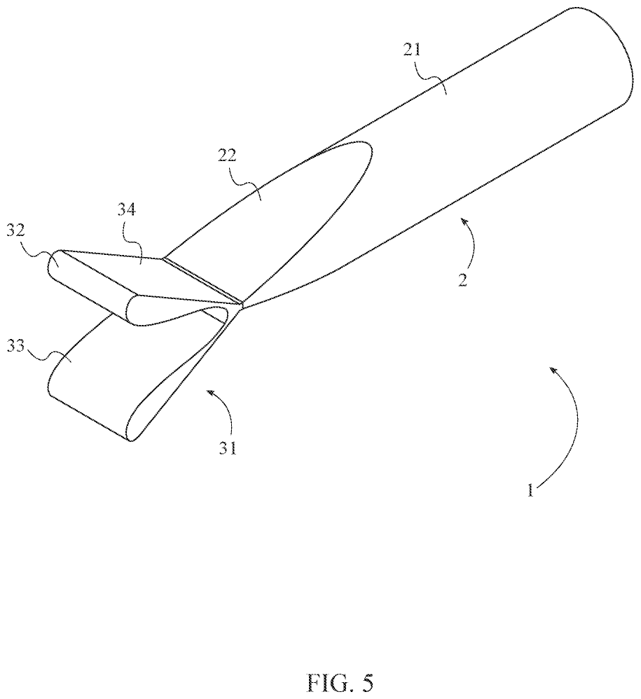

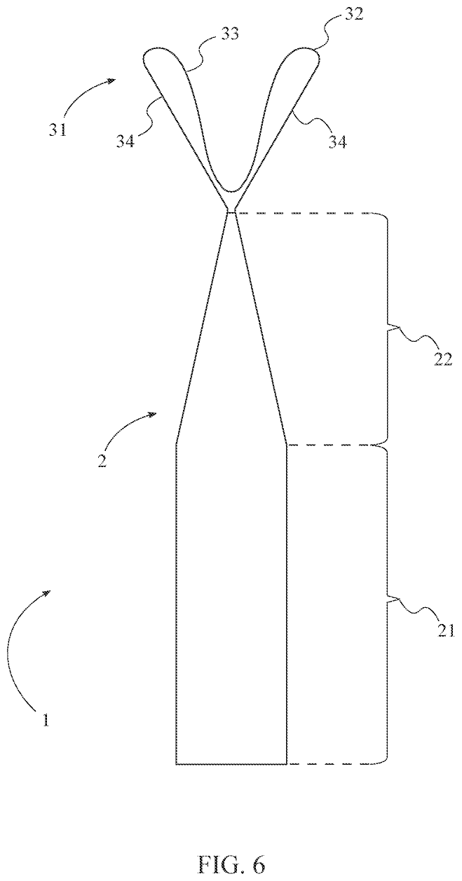

In order to properly pick up the contact lens tool 1, the handle 2 further comprises a tail region 21 and a head region 22. As best seen in FIGS. 3-6, the handle 2 extends from the tail region 21 to the head region 22. The tail region 21 constitutes a portion of the contact lens tool 1 that the user holds. The head region 22 constitutes a portion of the contact lens tool 1 that connects to and supports the applicator head 3. In a preferred embodiment, as seen in FIG. 3 and FIG. 4, both the tail region 21 and the head region 22 are of the same geometric profile in the form of a flat and rectangular strip. This arrangement provides improved handling and storage. In a second embodiment, as shown in FIG. 5 and FIG. 6, the tail region 21 and the head region 22 are of different geometric profiles. In the second embodiment, the tail region 21 is cylindrical in shape and the head region 22 tapers from a cylindrical shape to a flat, rectangular strip. However, it is understood that both the tail region 21 and the head region 22 are capable of comprising any and all feasible shapes based on design, user and/or manufacturing requirements (e.g., cylindrical, planar, rounded, angular, cuboidal). Thus, the geometric profile of the handle 2 for the present invention is not limited to the profiles depicted throughout FIGS. 1-9.

In order to properly pick up the contact lens 4, the applicator head 3 further comprises a pair of arms 31, an interior surface 33, and an exterior surface 34. As best seen in FIG. 3, Each of the pair of arms 31 are terminally connected to the head region 22 and extend outward, away from the handle 2. The interior surface 33 is disposed perimetrically along the inner surface region of each of the pair of arms 31. The exterior surface 34 is disposed perimetrically along the outer surface region of each of the pair of arms 31, opposite of the interior surface 33. Each of the pair of arms 31 further comprises a tip 32. The tip 32 extends outward from the distal end of each of the pair of arms 31. In the preferred embodiment, the shape of the tip 32 is round. The rounded profile provides optimal contact surface between the applicator head 3 and the contact lens 4. However, the tip 32 is not limited to the rounded shape depicted throughout FIGS. 1-9. Thus, the tip 32 can be planar, bulbous, beveled, or any other feasible shape based on design, user and/or manufacturing requirements.

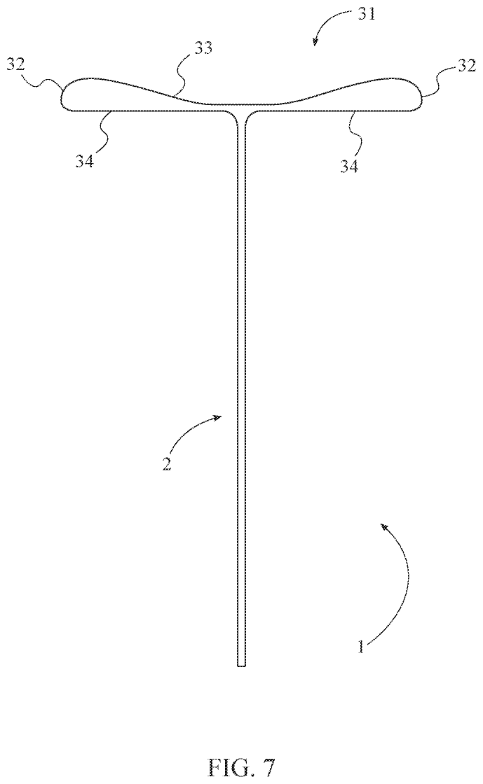

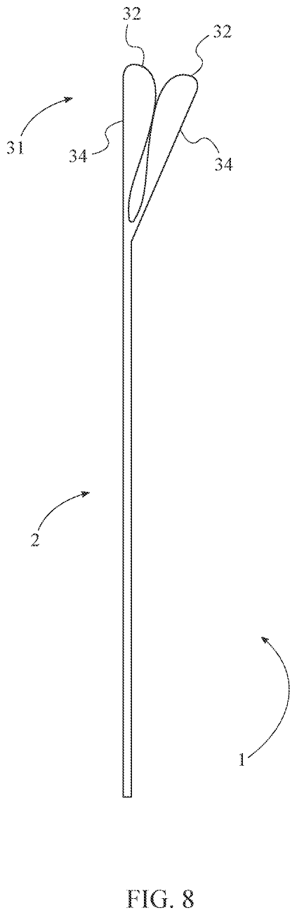

In the preferred embodiment, as best seen in FIG. 3, FIG. 7, and FIG. 8, the handle 2 and the applicator head 3 are produced and manufactured from a soft, moldable material that is reusable or disposable, such as PVC or any other suitable material capable of being easily reshaped. More specifically, the characteristics of such material allow the contact lens tool 1 to change shape when an external force is applied and retain this altered shape even after the external force is removed. As further detailed below, the ability to reshape the applicator head 3 into various positions provides enhanced effectiveness, depending on how the contact lens tool 1 is used.

Continuing with the preferred embodiment, when in use, each of the pair of arms 31 can be adjusted to form an open “Y” shape position, as best seen in FIG. 3. In other words, each of the pair of arms 31 are positioned to form an open angle with respect to each other. The “Y” shape is most effective for accommodating different contact lens characteristics. For example, the characteristics of soft contact lenses from the same manufacturer can vary depending on its power. A −6.00 Diopter contact lens is flat and tends to remain on the user's finger during insertion. In contrast, a +6.00 Diopter contact lens has the tendency to roll around and fall, making it difficult for insertion. Thus, the “Y” shape configuration provides improved handling by enabling the applicator head 3 to accommodate a wide range of contact lenses, regardless of their physical properties. Moreover, the “Y” shape configuration helps facilitate the proper contact lens orientation for insertion by exposing the posterior surface 42 of the contact lens 4 to the anterior surface of the cornea 5, as illustrated in FIG. 2. Alternatively, as seen in FIG. 7, each of the pair of arms 31 can be adjusted to be perpendicular with the handle 2, thereby forming a fully open “T” shape.

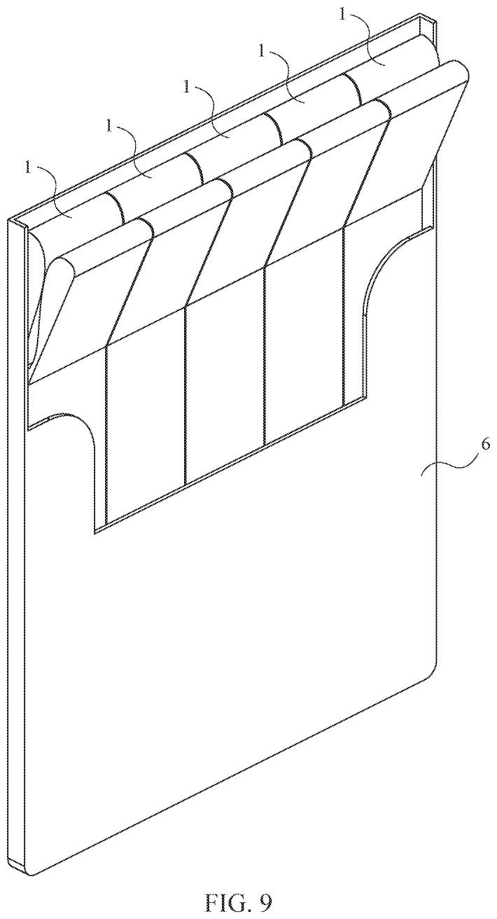

Continuing with the preferred embodiment, when not in use, each of the pair of arms 31 can be positioned parallel with the handle 2, thereby forming a fully closed “I” shape, as best seen in FIG. 8. In other words, each of the pair of arms 31 are positioned adjacent to each other, forming a relatively thin and flat profile. This configuration reduces the amount of space needed for storing the contact lens tool 1 when not in use. The “I” shape configuration is most effective for storing multiple contact lens tools 1 together inside a container 6, as seen in FIG. 9. The container 6 is not limited to the shape as seen in FIG. 9 and is capable of comprising any and all feasible shapes based on design, user and/or manufacturing requirements. The container 6 provides convenient storage, allowing the user to easily carry multiple contact lens tools 1 with them in their purse or wallet without altering the shape of the contact lens tool 1 or the shape of the user's purse or wallet. Alternatively, the container 6 can be in the form of product packaging, allowing the user to purchase the contact lens tool 1 in multiple quantities as a single, packaged item or included as part of another packaged product. When needed, the user can simply remove one contact lens tool 1 out of the container 6, adjust the pair of arms 31 to the desired shape, and use the contact lens tool 1 to insert or remove the contact lens 4.

In a third embodiment, as best seen in FIG. 3, the handle 2 and the applicator head 3 are produced and manufactured from a homogeneous, rigid material, such as polycarbonate or any other suitable material that is non-flexible. In this embodiment, each of the pair of arms 31 are positioned at an obtuse, acute, or right angle from each other, forming an open “Y” shape. More specifically, each of the pair of arms 31 are opened to an angle that is incident with the angle of the cornea's radius of curvature. This arrangement helps facilitate the proper contact lens orientation for insertion by exposing the posterior surface 42 of the contact lens 4 to the cornea 5. Added texture uses friction from the applicator head 3 to enhances removal of the contact lens 4. To prevent suction, the open “Y” shape can be of any form other than conical.

In a fourth embodiment, as seen in FIG. 7, the handle 2 and the applicator head 3 are produced and manufactured from a rigid material such as polycarbonate or any other suitable material that is non-flexible. In this embodiment, each of the pair of arms 31 are positioned opposite to each other and perpendicular to the handle 2, thereby forming a fully open “T” shape. This arrangement helps facilitate the proper contact lens orientation for insertion by exposing the posterior surface 42 of the contact lens 4 to the cornea 5.

Although the invention has been explained in relation to its preferred embodiment, it is to be understood that many other possible modifications and variations can be made without departing from the spirit and scope of the invention.

What is claimed is:

1. A contact lens tool comprising:
a handle;
an applicator head;
the handle comprising a tail region and a head region;
the handle extending from the tail region to the head region;
the applicator head comprising a pair of arms, an interior surface, and an exterior surface;
each of the pair of arms being terminally connected to the head region;
each of the pair of arms extending outward;
the interior surface being disposed perimetrically between each of the pair of arms; and
the exterior surface being disposed perimetrically on each of the pair of arms, opposite of the interior surface.

2. The contact lens tool as claimed in claim 1, wherein the handle and the applicator head are made of soft, moldable material.

3. The contact lens tool as claimed in claim 1, wherein the handle and the applicator head are made of rigid, non-moldable material.

4. The contact lens tool as claimed in claim 3, wherein each of the pair of arms are positioned at an open angle with each other.

5. The contact lens tool as claimed in claim 3, wherein each of the pair of arms are positioned opposite to each other and perpendicular to the handle.

6. The contact lens tool as claimed in claim 1 comprising:
the tail region and the head region being a continuous, rectangular shape.

7. The contact lens tool as claimed in claim 1 comprising:
the tail region being cylindrical in shape; and
the head region being rectangular in shape.

8. The contact lens tool as claimed in claim 1 comprising:
each of the pair of arms comprising a tip;
the tip being terminally connected to each of the pair of arms; and
the tip extending longitudinally outward from each of the pair of arms.

9. The contact lens tool as claimed in claim 8, wherein the tip comprising rounded edges.

10. A contact lens tool comprising:
a handle;
an applicator head;
the handle and the applicator head being made of soft, moldable material;
the handle comprising a tail region and a head region;
the handle extending from the tail region to the head region;
the applicator head comprising a pair of arms, an interior surface, and an exterior surface;
each of the pair of arms being terminally connected to the head region;
each of the pair of arms extending outward;
the interior surface being disposed perimetrically between each of the pair of arms; and
the exterior surface being disposed perimetrically on each of the pair of arms, opposite of the interior surface.

11. The contact lens tool as claimed in claim 10 comprising:
the tail region and the head region being a continuous, rectangular shape.

12. The contact lens tool as claimed in claim 10 comprising:
the tail region being cylindrical in shape; and
the head region being rectangular in shape.

13. The contact lens tool as claimed in claim 10 comprising:
each of the pair of arms comprising a tip;
the tip being terminally connected to each of the pair of arms; and
the tip extending longitudinally outward from each of the pair of arms.

14. The contact lens tool as claimed in claim 13, wherein the tip comprising rounded edges.

15. A contact lens tool comprising:
a handle;
an applicator head;
the handle and the applicator head being made of rigid, homogeneous, non-moldable material;
the handle comprising a tail region and a head region;
the handle extending from the tail region to the head region;
the applicator head comprising a pair of arms, an interior surface, and an exterior surface;
each of the pair of arms being terminally connected to the head region;
each of the pair of arms extending outward;
the interior surface being disposed perimetrically between each of the pair of arms; and
the exterior surface being disposed perimetrically on each of the pair of arms, opposite of the interior surface.

16. The contact lens tool as claimed in claim 15, wherein each of the pair of arms are positioned at an open angle with each other.

17. The contact lens tool as claimed in claim 15, wherein each of the pair of arms are positioned at an acute angle with each other.

18. The contact lens tool as claimed in claim 15, wherein each of the pair of arms are positioned orthogonal to each other.

19. The contact lens tool as claimed in claim 15 comprising:
each of the pair of arms comprising a tip;
the tip being terminally connected to each of the pair of arms; and
the tip extending longitudinally outward from each of the pair of arms.

20. The contact lens tool as claimed in claim 19, wherein the tip comprising beveled edges.
